(19) United States
(12) Patent Application Publication
(10) Pub. No.: US 2025/0051491 A1
(43) Pub. Date: Feb. 13, 2025

(54) ETHYLENE OLIGOMERIZATION CATALYST INCLUDING LIGAND COMPOUND, CATALYST COMPOSITION EMPLOYING SAME, AND METHOD FOR PRODUCING ETHYLENE OLIGOMER BY USING SAME

(71) Applicant: KOREA RESEARCH INSTITUTE OF CHEMICAL TECHNOLOGY, Daejeon (KR)

(72) Inventors: Dong Won HWANG, Daejeon (KR); Changho YOO, Daejeon (KR); Seung Hyeok CHA, Daejeon (KR); Gwang Nam YUN, Daejeon (KR)

(73) Assignee: KOREA RESEARCH INSTITUTE OF CHEMICAL TECHNOLOGY, Daejeon (KR)

(21) Appl. No.: 18/710,904

(22) PCT Filed: Mar. 27, 2023
(86) PCT No.: PCT/KR2023/004037

§ 371 Date: May 16, 2024

(30) Foreign Application Priority Data
Jul. 4, 2022 (KR) 10-2022-0082023
Mar. 24, 2023 (KR) 10-2023-0038809

(51) Int. Cl. C08F 4/78; C08F 110/02

(52) U.S. Cl. CPC C08F 4/78; C08F 110/02

(57) ABSTRACT

The present invention pertains to: an ethylene oligomerization catalyst containing a ligand compound; a catalyst composition employing same; and a method for producing an ethylene oligomer by using same. The catalyst exhibits excellent selectivity to oligomers and selectivity to 1-hexane, thus making it possible to mass-produce 1-hexane, which is an industrially useful chemical raw material, with high purity.

### TECHNICAL FIELD

The present invention relates to an ethylene oligomerization catalyst including a ligand compound, a catalyst composition employing the same, and a method for preparing an ethylene oligomer using the same.

### BACKGROUND ART

Olefin oligomers from ethylene are used as raw materials of various and useful chemical products. Specifically, higher olefins may be provided using olefin oligomers as raw materials, and industrially important processes include preparation of alpha olefins from ethylene. In particular, among alpha olefins prepared from ethylene, preparation of 1-hexene by trimerization of ethylene is very important.

However, a problem encountered in oligomerization is low selectivity to a single oligomer. That is, undesired side products such as other oligomers and polymers, and by-products may be formed with a desired oligomer. For example, when ethylene is oligomerized to produce 1-hexene, 2-hexene and 3-hexene which are other isomers of hexene may be formed, and also octene, decene, and dodecene which are higher oligomers of ethylene may be produced and other polyethylenes having a higher molecular weight may be formed.

The side products and by-products produced as such may cause problems in the process or efficiency. Since the produced polymer may be deposited in internal components, pipelines, and/or other equipment of an oligomerization reactor, a period of shutdown of process equipment may be needed for removing the deposited polymer, and a period and cost for physical treatment such as steam treatment and water treatment for removal may be consumed. In addition, due to an additional process and time taken for separating the side products and the by-products produced with the desired oligomer, production efficiency may be very lowered.

Meanwhile, an olefin oligomerization reaction from ethylene may be performed in an aliphatic hydrocarbon solvent. For example, cyclohexane, methyl cyclohexane, hexane, and heptane may be used in processes for preparing 1-hexene, and these solvents may have excellent solubility in an organic metal catalyst, but show a boiling point similar to that of 1-hexene, and thus, problems may arise in terms of cost and time, such as raising distillation stages and consuming a lot of calories in separation.

In order to meet needs for high single alpha olefin selectivity, in particular, high 1-hexene selectivity, new processes have been developed. A selective C6-commercialization process created by Chevron Phillips (see J. T. Dixon, M. J. Green, F. M. Hess, D. H. Morgan, “Advances in selective ethylene trimerisation—a critical overview”, Journal of Organometallic Chemistry 689 (2004) 3641-3668) is known, and a patent application (WO 03/053891 A1) filed by Sasol discloses a chromium-based selective ethylene-trimerization catalyst systems in the form of traditional CrCl₃ (bis-(2-diphenylphosphino-ethyl)amine)/methyl aluminoxane (MAO). However, all catalyst systems as such produce a significant amount of side products and by-products such as polyethylene and other alpha olefins, in addition to 1-hexene.

That is, a need for a catalyst system having increased selectivity and high efficiency of alpha olefin for a desired olefin isomer still remains. Therefore, a study of a catalyst which may achieve improved selectivity, decreased formation of side products and by-products, an improved yield of a desired alpha olefin, improved economic feasibility, and improved efficiency and a preparation method thereof is needed.

### DISCLOSURE

### Technical Problem

An object of the present invention is to provide an ethylene oligomerization catalyst including a ligand compound showing excellent 1-hexene selectivity.

Another object of the present invention is to provide an ethylene oligomerization catalyst composition having excellent activity and improved selectivity even at a high temperature employing the catalyst.

Still another object of the present invention is to provide a method for preparing a commercially available ethylene oligomer using the catalyst composition.

### Technical Solution

In one general aspect, an ethylene oligomerization catalyst includes: a chromium compound and a ligand compound represented by the following Chemical Formula 1:

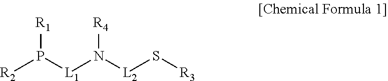

wherein
L₁ and L₂ are independently of each other (C2-C4)alkylene;
R₁ to R₄ are independently of one another hydrogen, (C1-C10)alkyl, (C3-C20)cycloalkyl, (C6-C20)aryl, (C6-C20)aryl(C1-C10)alkyl, or (C1-C10)alkoxy; and
the aryl of R₁ to R₄ may be substituted by one or two or more substituents selected from the group consisting of (C1-C10)alkyl, (C6-C20)aryl, tri(C1-C10)alkylsilyl, and (C1-C10)alkoxy.

Specifically, in Chemical Formula 1, L₁ and L₂ may be independently of each other (C2-C3)alkylene; R₁ to R₄ may be independently of one another hydrogen, (C1-C7)alkyl, (C3-C12)cycloalkyl, (C6-C12)aryl, (C6-C12)aryl(C1-C7)alkyl, or (C1-C7)alkoxy; and the aryl of R₁ to R₄ may be substituted by one or two or more substituents selected from the group consisting of (C1-C7)alkyl, (C6-C12)aryl, tri(C1-C7)alkylsilyl, and (C1-C7)alkoxy.

More specifically, in Chemical Formula 1, L₁ and L₂ may be independently of each other (C2-C3)alkylene; R₁ to R₄ may be independently of one another hydrogen, (C1-C7)alkyl, (C6-C12)aryl, or (C6-C12)aryl(C1-C7)alkyl; and the aryl of R₁ to R₄ may be substituted by one or two or more substituents selected from the group consisting of (C1-C7)alkyl, (C6-C12)aryl, tri(C1-C7)alkylsilyl, and (C1-C7)alkoxy.

The ligand compound according to an exemplary embodiment of the present invention may be represented by the following Chemical Formula 2:

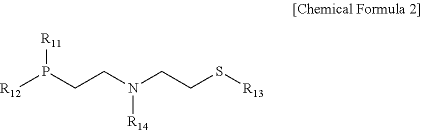

wherein
R₁₁ to R₁₄ are independently of one another hydrogen, (C1-C5)alkyl, (C6-C10)aryl, or (C6-C10)aryl(C1-C5)alkyl; and
the aryl of R₁₁ to R₁₄ may be substituted by one or two or more substituents selected from the group consisting of (C1-C5)alkyl, tri(C1-C5)alkylsilyl, and (C1-C5)alkoxy.

In addition, in Chemical Formula 2, R₁₁ to R₁₃ may be independently of one another (C1-C5)alkyl, (C6-C10)aryl, or (C6-C10)aryl(C1-C5)alkyl; R₁₄ may be hydrogen or (C1-C3)alkyl; and the aryl of R₁₁ to R₁₃ may be substituted by one or two or more substituents selected from the group consisting of (C1-C5)alkyl, tri(C1-C5)alkylsilyl, and (C1-C5)alkoxy.

Specifically, the ligand compound according to an exemplary embodiment of the present invention may be selected from the following compounds:

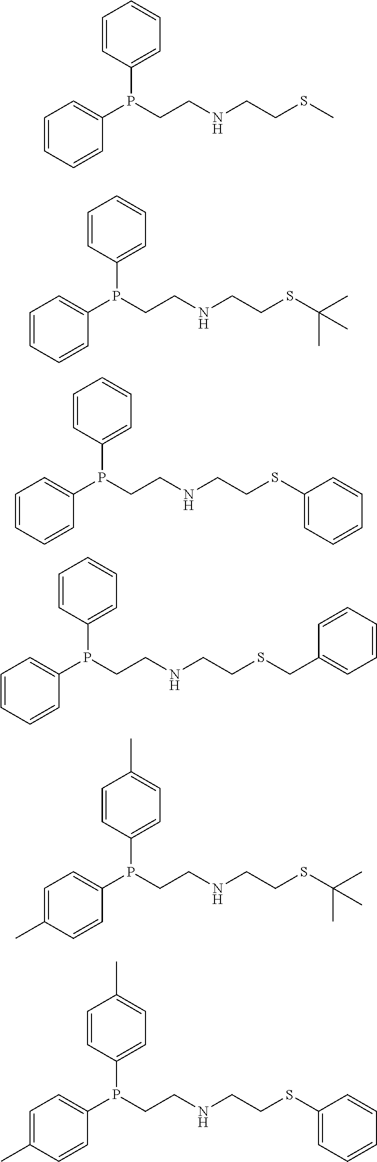

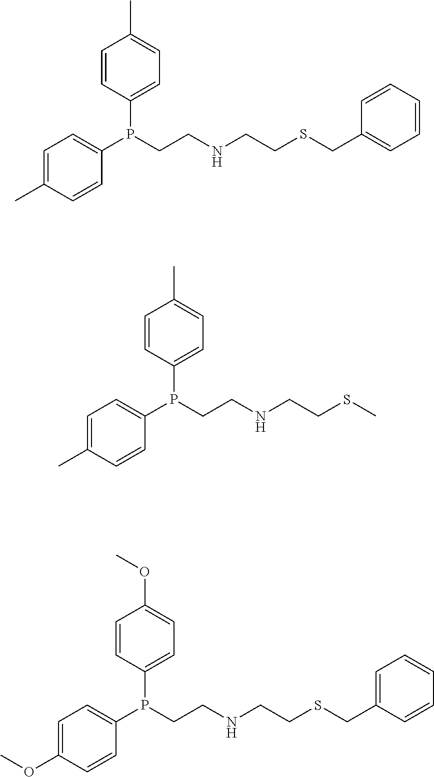

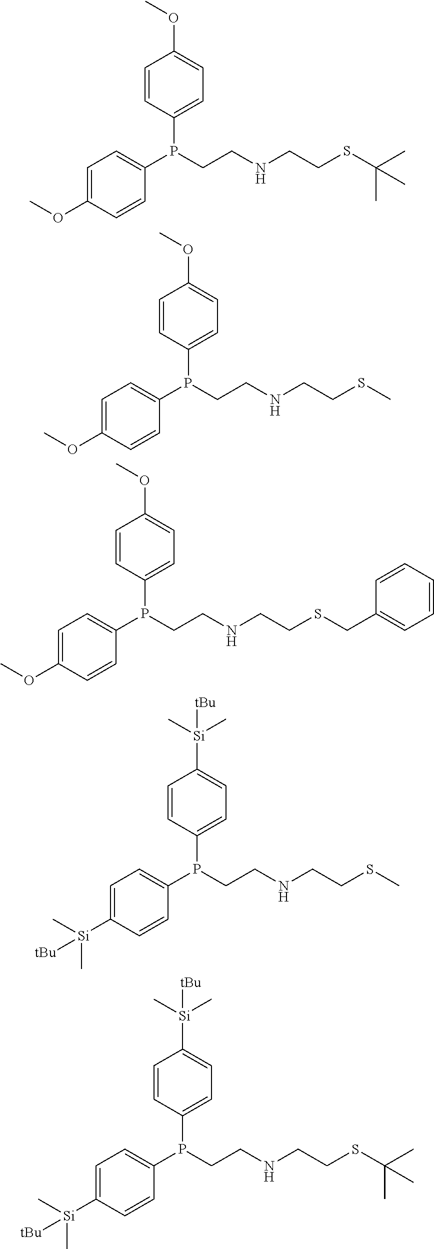

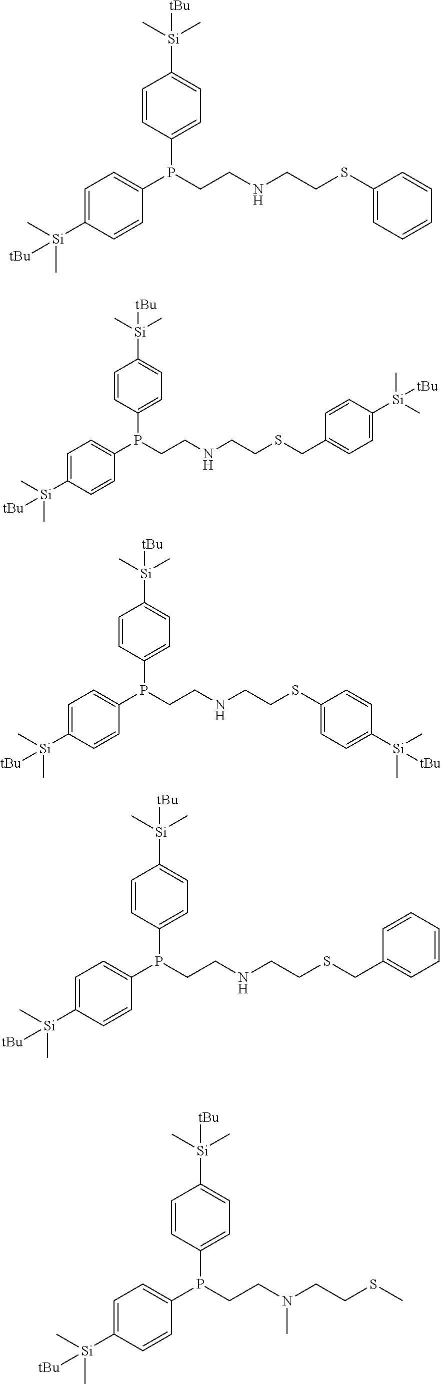

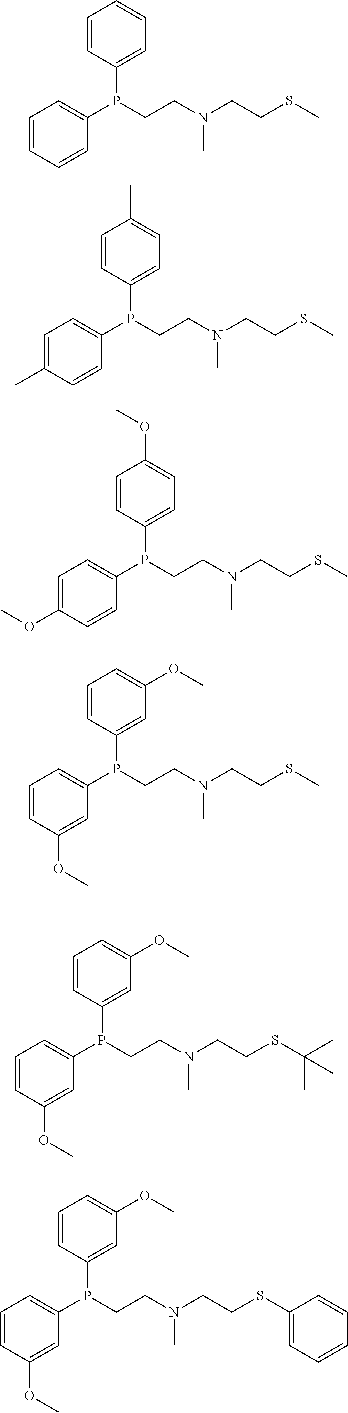

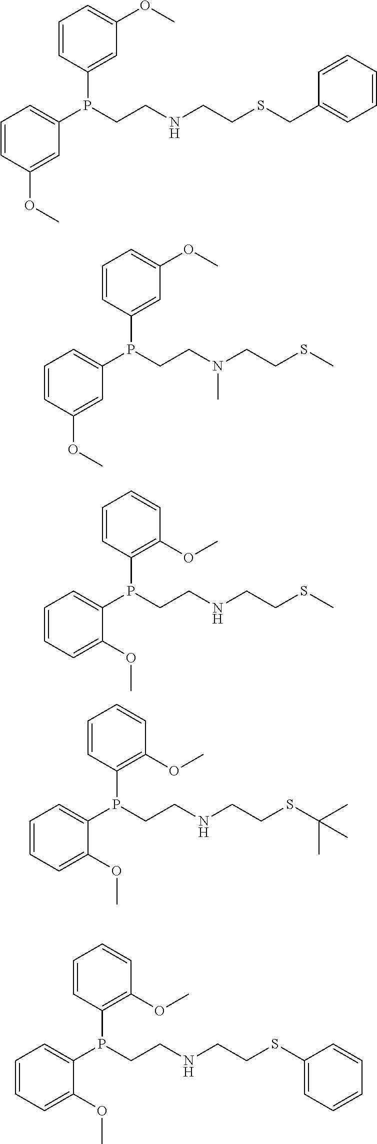

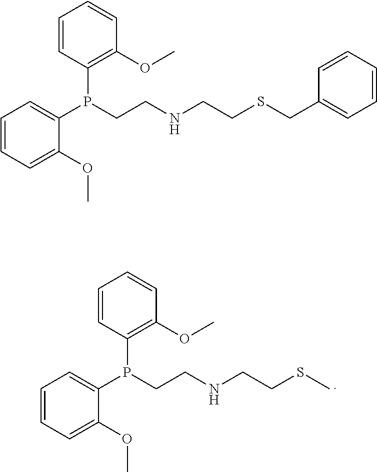 [SMILES: COc1ccccc1P(CCNCCSCc1ccccc1)c1ccccc1OC ; COc1ccccc1P(CCNCCSC)c1ccccc1OC]

In another general aspect, an ethylene oligomerization catalyst composition includes: the ethylene oligomerization catalyst according to an exemplary embodiment and an organoaluminum compound.

The organoaluminum compound may be one or two or more selected from the group consisting of methylaluminoxane (MAO), modified methylaluminoxane (MMAO), ethylaluminoxane (EAO), tetraisobutylaluminoxane (TIBAO), isobutylaluminoxane (IBAO), trimethylaluminum (TMA), triethylaluminum (TEA), triisobutylaluminum (TIBA), tri-n-octylaluminum, methylaluminum dichloride, ethylaluminum dichloride, dimethylaluminum chloride, diethylaluminum chloride, aluminum isopropoxide, ethylaluminum sesquichloride, and methylaluminum sesquichloride.

In still another general aspect, a method for preparing an ethylene oligomer using the ethylene oligomerization catalyst composition according to an exemplary embodiment is provided.

The method for preparing an ethylene oligomer may produce 60.0 to 99.999 wt % of 1-hexene of the total oligomer produced.

In addition, in an exemplary embodiment, the method for preparing an ethylene oligomer may be performed in one or two or more solvents selected from the group consisting of benzene, chlorobenzene, ethylbenzene, toluene, xylene, cumene, mesitylene, cyclohexane, methylcyclohexane, methylcyclopentane, hexane, heptane, octane, nonane, decane, hexene, heptene, octene, nonene, decene, anisole, ethoxybenzene, and dimethoxybenzene.

### Advantageous Effects

The ethylene oligomerization catalyst including the specific ligand compound of the present invention and the catalyst composition employing the catalyst may show improved selectivity to 1-hexene.

The method for preparing an ethylene oligomer using the catalyst composition of the present invention shows surprisingly excellent selectivity to an oligomer and selectivity to 1-hexene even at a high temperature, and thus, may mass produce 1-hexene which is an industrially useful chemical raw material with high purity.

### BEST MODE

Hereinafter, an ethylene oligomerization catalyst including a ligand compound of the present invention, a catalyst composition employing the same, and a method for preparing an ethylene oligomer using the same will be described in detail.

The singular form used in the present invention may be intended to also include a plural form, unless otherwise indicated in the context.

The term “comprise” described in the present invention is an open-ended description having a meaning equivalent to the term such as “is/are provided”, “contain”, “have”, or “is/are characterized”, and does not exclude elements, materials or processes which are not further listed.

“Alkyl” described in the present invention refers to a monovalent straight-chain or branched-chain saturated hydrocarbon radical consisting of only carbon and hydrogen atoms, and an example of the alkyl radical includes methyl, ethyl, propyl, isopropyl, butyl, isobutyl, t-butyl, pentyl, hexyl, octyl, nonyl, or the like, but is not limited thereto.

“Aryl” described in the present invention refers to an organic radical derived from an aromatic hydrocarbon by removal of one hydrogen, including a single- or fused ring system containing appropriately 4 to 7, preferred 5 or 6 ring atoms in each ring, and even a form in which a plurality of aryls are linked by a single bond. A fused ring system may include an aliphatic ring such as saturated or partially saturated rings, and necessarily includes one or more aromatic rings. In addition, the aliphatic ring may contain nitrogen, oxygen, sulfur, carbonyl, and the like in the ring. The specific example of the aryl radical includes phenyl, naphthyl, biphenyl, indenyl, fluorenyl, phenanthrenyl, anthracenyl, triphenylenyl, pyrenyl, chrysenyl, naphthacenyl, 9,10-dihydroanthracenyl, and the like, but is not limited thereto.

“Cycloalkyl” described in the present invention refers to a monovalent saturated carbocyclic radical formed of one or more rings. An example of the cycloalkyl radical includes cyclopropyl, cyclobutyl, cyclopentyl, cyclohexyl, cycloheptyl, and the like, but is not limited thereto.

“Alkoxy” described in the present invention refers to —O-(alkyl) including —OCH₃, —OCH₂CH₃, —O(CH₂)₂CH₃, —O(CH₂)₃CH₃, —O(CH₂)₄CH₃, —O(CH₂)₅CH₃, and the like, in which “alkyl” is as defined above.

An example of “trialkylsilyl” described in the present invention may include a group in which three hydrogens in a silyl group are independently of each other substituted by alkyl, in which “alkyl” is as defined above. A preferred alkyl to be substituted is an alkyl having 1 to 5 carbon atoms and an example thereof may be specifically methyl, ethyl, propyl, isopropyl, butyl, sec-butyl, tert-butyl, cyclobutyl, and the like, and more specifically, trialkylsilyl may be trimethylsilyl, triethylsilyl, t-butyldimethylsilyl, vinyldimethylsilyl, or propyldimethylsilyl, but is not limited thereto.

“Halogen” described in the present invention refers to fluorine, chlorine, bromine, or an iodine atom.

“Ethylene oligomerization” described in the present invention is oligomerization of ethylene, and may be referred to as trimerization and tetramerization depending on the number of ethylene to be polymerized. In particular, in the present invention, it refers to preparation of 1-hexene obtained by trimerization of ethylene used as a comonomer of HDPE and LLDPE.

The present invention provides an ethylene oligomerization catalyst including: a chromium compound and a ligand compound represented by the following Chemical Formula 1:

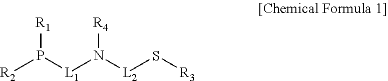

wherein
L₁ and L₂ are independently of each other (C2-C4)alkylene;
R₁ to R₄ are independently of one another hydrogen, (C1-C10)alkyl, (C3-C20)cycloalkyl, (C6-C20)aryl, (C6-C20)aryl(C1-C10)alkyl, or (C1-C10)alkoxy; and
the aryl of R₁ to R₄ may be substituted by one or two or more substituents selected from the group consisting of (C1-C10)alkyl, (C6-C20)aryl, tri(C1-C10)alkylsilyl, and (C1-C10)alkoxy.

The ethylene oligomerization catalyst of the present invention may be in a complex form in which the ligand compound of Chemical Formula 1 is coordinated to the chromium compound as a chromium source or a composition form including the chromium compound and the ligand compound of Chemical Formula 1.

In a specific example, the ethylene oligomerization catalyst may be a complex having a structure of (L)CrX₃ wherein L is the ligand compound of Chemical Formula 1, and X is halogen, C1-C10 alkylcarbonyloxy, haloC1-C10 alkylcarbonyloxy, acetylacetonate, pyrrolide, pyrazolide, imidazolide, 1,2,3-triazolide, tetrazolide, or indolide.

The chromium compound may be specifically a chromium trivalent compound, and may be, for example, one or two or more selected from the group consisting of chromium (III) chloride (CrCl₃), chromium (III) acetate (Cr(OAc)₃), chromium (III) 2-ethylhexanoate (Cr(EH)₃), chromium (III) acetylacetonate (Cr(acac)₃), and chromium (III) pyrolide.

The ethylene oligomerization catalyst of the present invention includes a specific ligand showing a structure of P—N—S, thereby showing high activity even at a high temperature, having excellent selectivity to an oligomer compared with polymers, and having significantly improved selectivity of the oligomer to 1-hexene, and thus, may produce 1-hexene with excellent yield and selectivity.

In Chemical Formula 1 according to an exemplary embodiment, L¹ and L² may be independently of each other (C2-C3)alkylene; R₁ to R₄ may be independently of one another hydrogen, (C1-C7)alkyl, (C3-C12)cycloalkyl, (C6-C12)aryl, (C6-C12)aryl(C1-C7)alkyl, or (C1-C7)alkoxy; and the aryl of R₁ to R₄ may be substituted by one or two or more substituents selected from the group consisting of (C1-C7)alkyl, (C6-C12)aryl, tri(C1-C7)alkylsilyl, and (C1-C7)alkoxy.

Specifically, in Chemical Formula 1, L₁ and L₂ may be independently of each other (C2-C3)alkylene; R₁ to R₄ may be independently of one another hydrogen, (C1-C7)alkyl, (C6-C12)aryl, or (C6-C12)aryl(C1-C7)alkyl; and the aryl of R₁ to R₄ may be substituted by one or two or more substituents selected from the group consisting of (C1-C7)alkyl, (C6-C12)aryl, tri(C1-C7)alkylsilyl, and (C1-C7)alkoxy.

More specifically, in Chemical Formula 1, L₁ and L₂ may be independently of each other (C2-C3)alkylene; R₁ to R₃ may be independently of one another (C1-C5)alkyl, (C6-C10)aryl, or (C6-C10)aryl(C1-C5)alkyl; R₁₄ may be hydrogen or (C1-C5)alkyl; and the aryl of R₁ to R₃ may be substituted by one or two or more substituents selected from the group consisting of (C1-C5)alkyl, (C6-C10)aryl, tri(C1-C5)alkylsilyl, and (C1-C5)alkoxy.

The ligand compound according to an exemplary embodiment of the present invention may be represented by the following Chemical Formula 2:

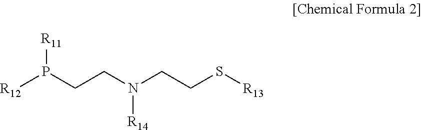

wherein
R₁₁ to R₁₄ are independently of one another hydrogen, (C1-C5)alkyl, (C6-C10)aryl, or (C6-C10)aryl(C1-C5)alkyl; and
the aryl of R₁ to R₁₄ may be substituted by one or two or more substituents selected from the group consisting of (C1-C5)alkyl, tri(C1-C5)alkylsilyl, and (C1-C5)alkoxy.

In addition, in Chemical Formula 2, R₁ to R₁₃ may be independently of one another (C1-C5)alkyl, (C6-C10)aryl, or (C6-C10)aryl(C1-C5)alkyl; R₁₄ may be hydrogen or (C1-C3)alkyl; and the aryl of R₁₁ to R₁₃ may be substituted by one or two or more substituents selected from the group consisting of (C1-C5)alkyl, tri(C1-C5)alkylsilyl, and (C1-C5)alkoxy.

In Chemical Formula 2 according to an exemplary embodiment of the present invention, R₁ to R₁₃ may be independently of one another (C1-C5)alkyl, phenyl, or benzyl; R₁₄ may be hydrogen or methyl; and the phenyl and the benzyl of R₁₁ to R₁₃ may be independently of one another substituted by one or two or more substituents selected from the group consisting of (C1-C5)alkyl, tri(C1-C5)alkylsilyl, and (C1-C3)alkoxy.

The ligand compound according exemplary embodiment of the present invention may be represented by the following Chemical Formula 3:

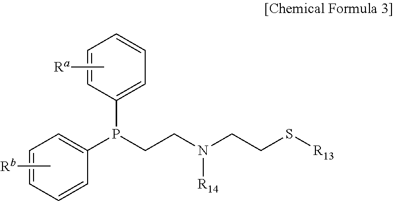

wherein
R₁₃ is independently of each other (C1-C5)alkyl, phenyl, or benzyl, and the phenyl of R₁₃ may be substituted by one or two or more substituents s selected from the group consisting of (C1-C5)alkyl, tri(C1-C5)alkylsilyl, and (C1-C3)alkoxy;
R₁₄ is hydrogen or methyl; and
R^{a} and R^{b} are independently of each other hydrogen, (C1-C5)alkyl, tri(C1-C5)alkylsilyl, or (C1-C3)alkoxy.

Specifically, the ligand compound according to an exemplary embodiment of the present invention may be selected from the following compounds:

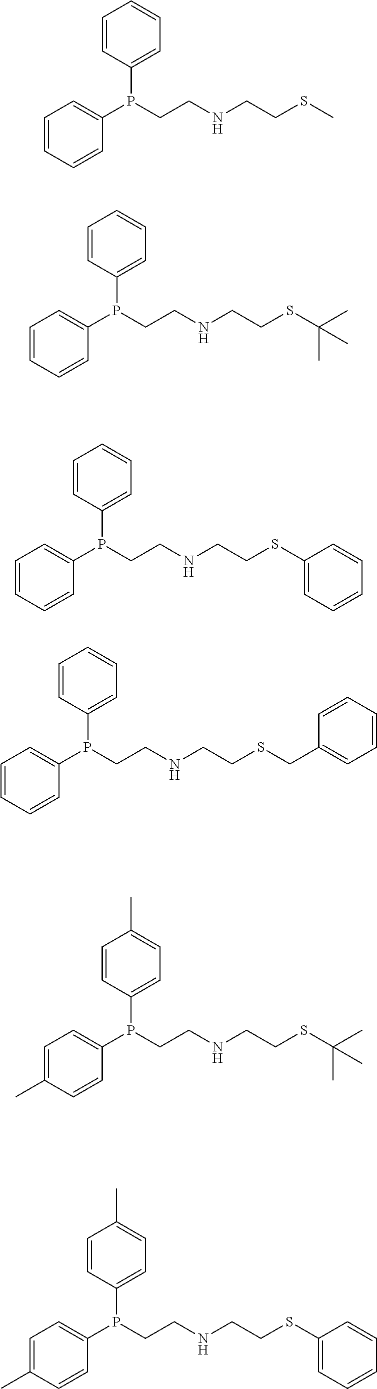

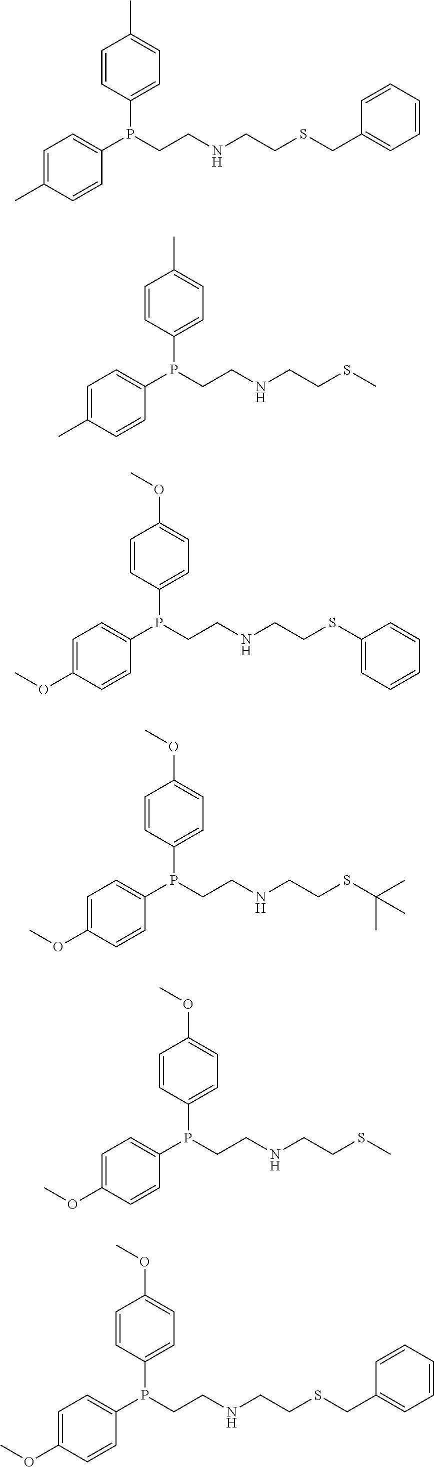

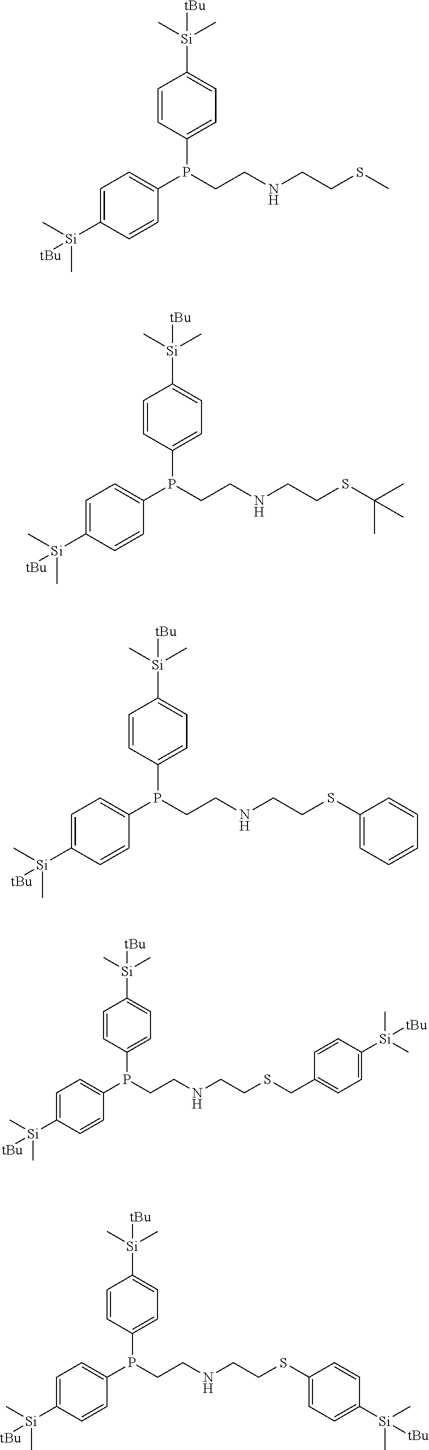

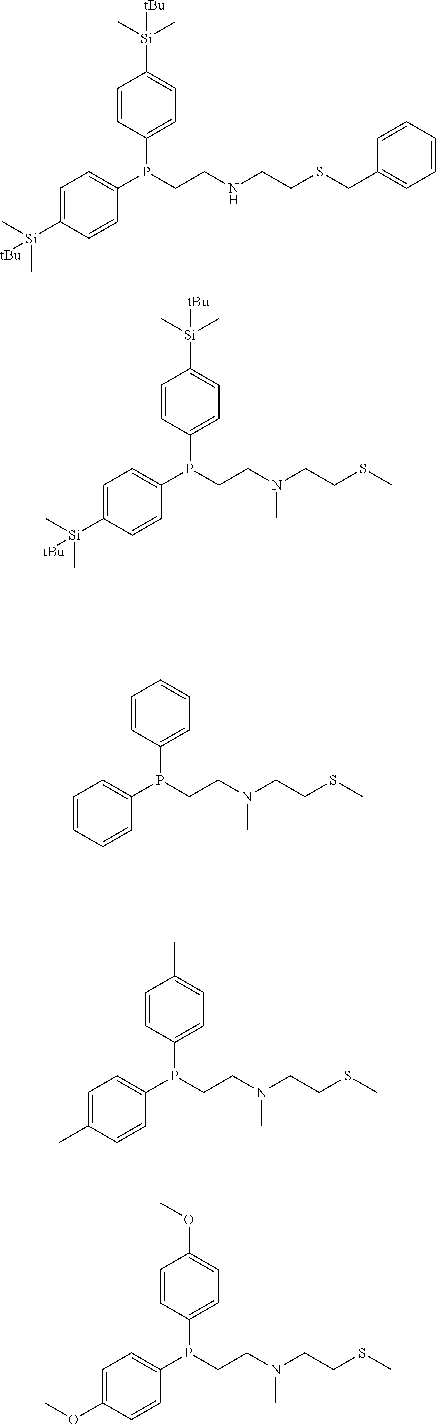

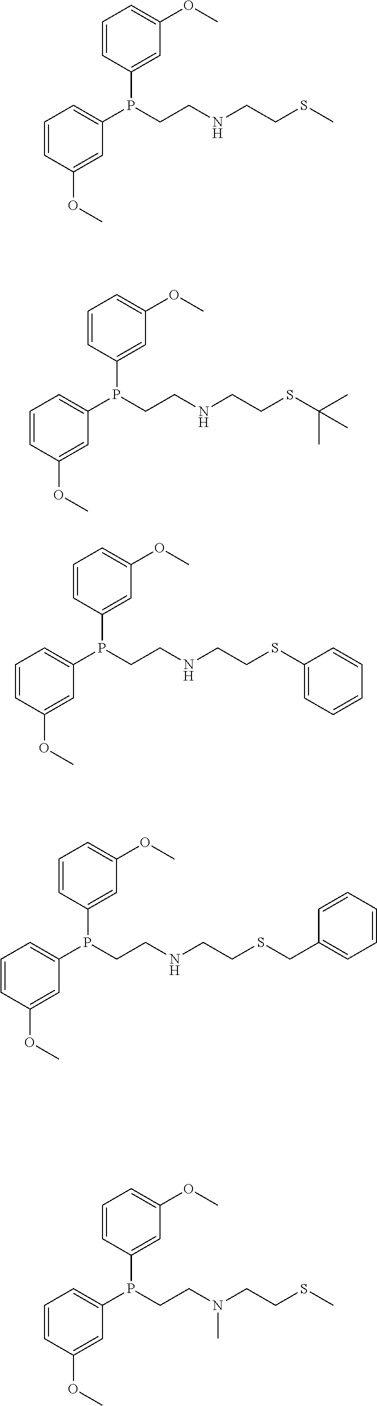

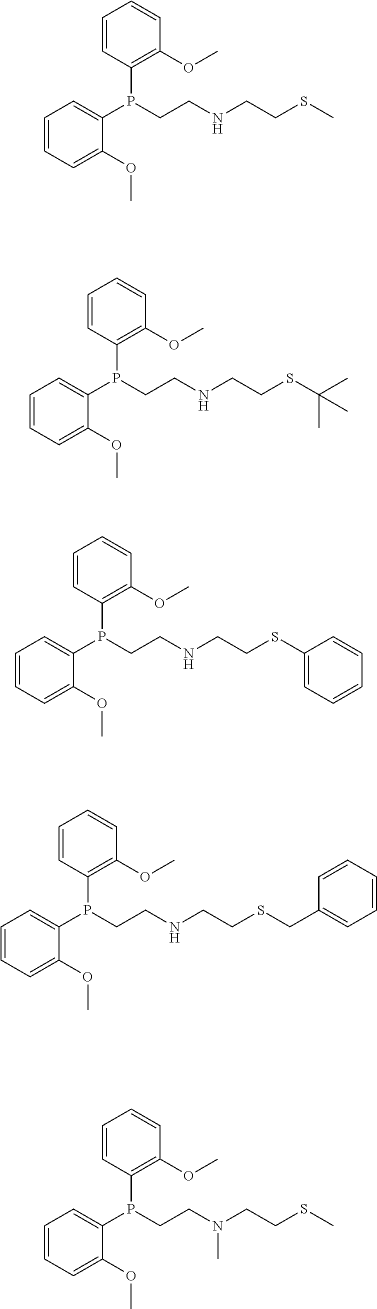 [SMILES: COc1ccccc1P(CCNCCSC)c1ccccc1OC ; COc1ccccc1P(CCNCCSC(C)(C)C)c1ccccc1OC ; COc1ccccc1P(CCNCCSc1ccccc1)c1ccccc1OC ; COc1ccccc1P(CCNCCSCc1ccccc1)c1ccccc1OC ; COc1ccccc1P(CCN(C)CCSC)c1ccccc1OC]

The present invention provides a method for preparing the specific ligand compound, and the ligand compound represented by the following Chemical Formula 1 may be prepared by reacting a compound of the following Chemical Formula 11 and a compound of the following Chemical Formula 12:

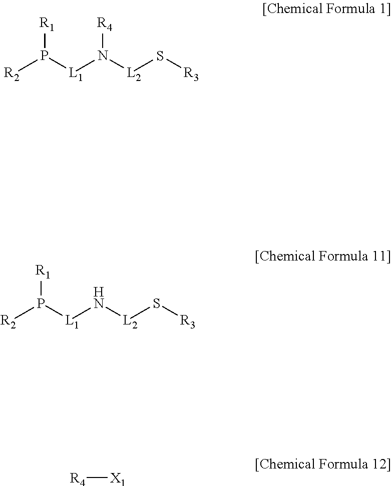

wherein
L₁ and L₂ are independently of each other (C2-C4)alkylene;
R₁ to R₄ are independently of one another (C1-C10)alkyl, (C3-C20)cycloalkyl, (C6-C20)aryl, (C6-C20)aryl(C1-C10)alkyl, or (C1-C10)alkoxy;
X₁ is a halogen; and
the aryl of R₁ to R₄ may be substituted by one or two or more substituents selected from the group consisting of (C1-C10)alkyl, (C6-C20)aryl, tri(C1-C10)alkylsilyl, and (C1-C10)alkoxy.

In addition, the compound represented by the following Chemical Formula 11 may be prepared by reacting a compound of the following Chemical Formula 13 and a compound of the following Chemical Formula 14:

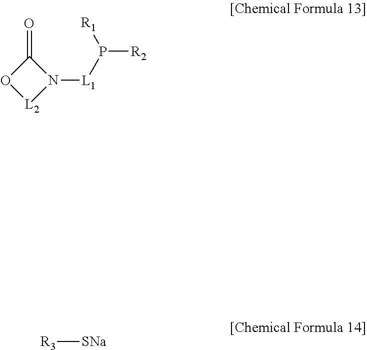

wherein L₁, L₂, R₁, R₂, and R₅ are as defined in Chemical Formula 1 above.

In addition, the compound of Chemical Formula 13 may be prepared by reacting a compound of the following Chemical Formula 15 and a compound of the following Chemical Formula 16:

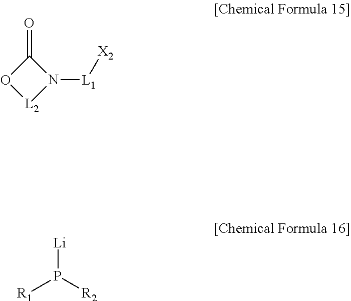

wherein L₁, L₂, R₁, and R₂ are as defined in Chemical Formula 1 above; and
X₂ is a halogen.

The method for preparing a ligand compound according to an exemplary embodiment of the present invention may easily secure a ligand having different chemical properties which has various substituents using a raw material compound having a changed substituent in the preparation process, and thus, may be favorable for activity adjustment and support of the catalyst.

The present invention provides an ethylene oligomerization catalyst composition including: the ethylene oligomerization catalyst according to an exemplary embodiment and an organoaluminum compound.

The ethylene oligomerization catalyst composition employing the ethylene oligomerization catalyst of the present invention shows excellent catalytic activity and very high selectivity to 1-hexene even at a high reaction temperature, and thus, may efficiently produce 1-hexene which is industrially useful as a chemical raw material.

The organoaluminum compound may be a compound of AlR₃ wherein R is independently of each other C1-C12 alkyl, C6-C20 aryl, C2-C10 alkenyl, C2-C10 alkynyl, C1-C12 alkoxy, or a halogen. Specifically, the organoaluminum compound is one or two or more selected from the group consisting of methylaluminoxane (MAO), modified methylaluminoxane (MMAO), ethylaluminoxane (EAO), tetraisobutylaluminoxane (TIBAO), isobutylaluminoxane (IBAO), trimethylaluminum (TMA), triethylaluminum (TEA), triisobutylaluminum (TIBA), tri-n-octylaluminum, methylaluminum dichloride, ethylaluminum dichloride, dimethylaluminum chloride, diethylaluminum chloride, aluminum isopropoxide, ethylaluminum sesquichloride, and methylaluminum sesquichloride, but is not limited thereto.

In an exemplary embodiment of the present invention, a ratio between the ethylene oligomerization catalyst and the organoaluminum compound may be represented by a mole ratio of chromium in the ethylene oligomerization catalyst:aluminum in the organoaluminum compound, which may be 1:1 to 10,000, preferably 1:1 to 2,000, and more preferably 1:1 to 1,000.

Individual component of the ethylene oligomerization catalyst composition may be simultaneously combined in the presence of a solvent or combined sequentially in any order. The combination may be performed at 20 to 250° C., preferably 20 to 160° C.

The ethylene oligomerization catalyst composition according to an exemplary embodiment of the present invention has very high catalytic activity and selectivity to 1-hexene, its input amount may be adjusted, and furthermore, its excellent activity remains constant even at a high temperature so that pipe blockage and fouling which are problems during an olefin preparation process do not occur, and thus, the composition is very economical and efficient.

In addition, the present invention provides a method for preparing an ethylene oligomer using the ethylene oligomerization catalyst composition according to an exemplary embodiment.

The method for preparing an ethylene oligomer according to an exemplary embodiment may be prepared by a homogeneous liquid phase reaction, a two-phase liquid/liquid reaction, a bulk phase reaction in which the product acts as a main medium, or a gas phase reaction in the presence of an inert solvent, using the oligomerization catalyst composition, a common device, and a contact technology, but preferably, may be prepared by a homogeneous liquid phase reaction in the presence of an inert solvent.

A polymer of the product prepared by the method for preparing an ethylene oligomer may be 0.001 wt % or more, 0.01 wt % or more, or 0.1 wt % or more and 40.0 wt % or less, 30.0 wt % or less, or 20.0 wt % or less, and for example, may be 0.001 to 30.0 wt %, specifically 0.01 to 30.0 wt %, and more specifically 0.1 to 20.0 wt %, of the total product.

The oligomer of the product prepared by the method for preparing an ethylene oligomer may be 60.0 wt % or more, 70.0 wt % or more, or 80.0 wt % or more and 99.999 wt % or less, 99.99 wt % or less, or 99.9 wt % or less, and for example, 70.0 to 99.999 wt %, specifically 70.0 to 99.99 wt %, and more specifically 80.0 to 99.9 wt %, of the total product.

In addition, 1-hexene prepared by the preparation method may be 60.0 wt % or more, 70.0 wt % or more, 80.0 wt % or more, or 90.0 wt % or more and 99.999 wt % or less, 99.99 wt % or less, or 99.9 wt % or less, and for example, 60.0 to 99.999 wt %, specifically 65.0 to 99.99 wt %, more specifically 70.0 to 99.9 wt %, and still more specifically 80 to 99.9 wt %, of total oligomer.

The method for preparing an ethylene oligomer using the catalyst composition of the present invention shows surprisingly excellent selectivity to an oligomer and selectivity to 1-hexene even at a high temperature, and thus, may mass produce 1-hexene which is an industrially useful chemical raw material with high purity.

In addition, in an exemplary embodiment, the method for preparing an ethylene oligomer may be performed in one or two or more solvents selected from the group consisting of aromatic hydrocarbons such as benzene, chlorobenzene, ethylbenzene, toluene, xylene, cumene, and mesitylene; cyclic aliphatic hydrocarbons such as cyclohexane, methylcyclohexane, methylcyclopentane; aliphatic hydrocarbons such as hexane, heptane, octane, nonane, and decane; olefins such as hexene, heptene, octene, nonene, and decene; and aromatic ethers such as anisole, ethoxybenzene, and dimethoxybenzene. Specifically, the solvent may be toluene, xylene, nonane, hexane, or a mixture thereof, and more specifically, toluene, nonane, or a mixture thereof.

The oligomerization reaction according to an exemplary embodiment of the present invention may be performed at −20 to 250° C., preferably 20 to 160° C., and more preferably 60 to 120° C.

In addition, the oligomerization reaction may be performed at a pressure of 1 to 100 bar, preferably 5 to 70 bar, and more preferably 10 to 40 bar.

The method for preparing an ethylene oligomer according to an exemplary embodiment of the present invention may be performed in a plant including an any type of reactor. The reactor may be, for example, batch, semi-batch, and continuous, but is not limited thereto. The plant may include a reactor, an olefin reactor inside the reactor, an inlet of the oligomerization catalyst composition, a line for outflow of the oligomerization reaction product from the reactor, and one or two or more separators for separating the oligomerization reaction product.

Hereinafter, the ethylene oligomerization catalyst including a ligand compound according to the present invention, the catalyst composition employing the same, and the method for preparing an ethylene oligomer using the same will be described in more detail, using the specific examples.

### [Preparation Example A] Preparation of Compound A

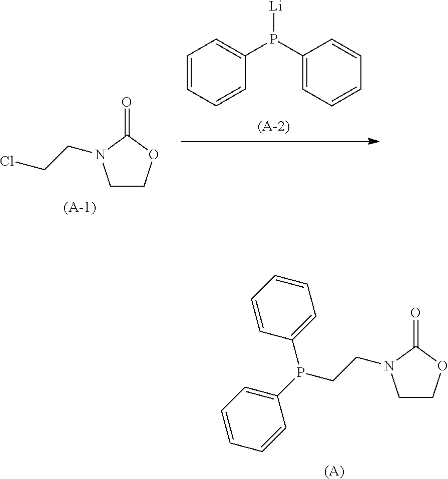

Compound A-2 (3.82 mL, 22 mmol) and 20 mL of dried THE were added to a dried flask under a nitrogen atmosphere and cooled to 5° C., and 2.5 M n-BuLi in Hex (8.8 ml, 22 mmol) was slowly added thereto. Stirring was performed at room temperature for 20 minutes, and then the flask was cooled to −78° C. Compound A-1 (3.0 g, 20 mmol) and 30 mL of dried THE were mixed at −78° C., and the mixed solution was slowly added for 1 hour. 100 mL of toluene and 25 mL of water were added at room temperature, and distilled under reduced pressure. An organic layer was washed, moisture was removed with MgSO₄, recrystallization was performed with 2-methyl-2-butanol, and then vacuum drying was performed to obtain Compound A (yield: 51%).

¹H NMR (CDCl₃, 500 MHZ) δ 7.50-7.28 (m, 10H), 4.23-4.19 (m, 2H), 3.56-3.43 (m, 4H), 2.39-2.35 (m, 2H)

### [Preparation Example B] Preparation of Compound B

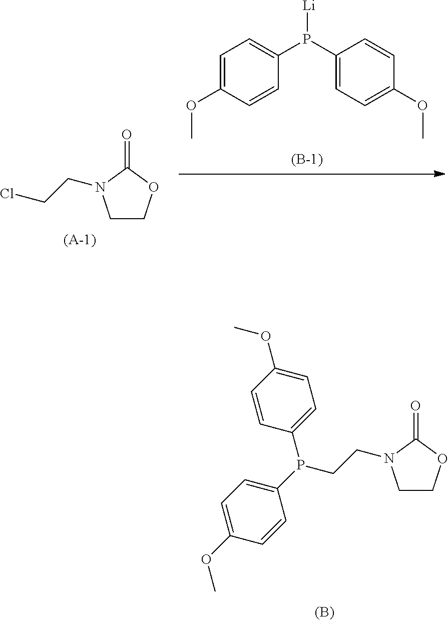

Compound B was obtained (yield: 54%) in the same manner as in Preparation Example A, except that Compound B-1 was used instead of Compound A-2.

¹H NMR (CDCl₃, 500 MHZ) δ 7.38-7.34 (m, 4H), 6.88 (d, 4H, J=7.8 Hz), 4.18 (t, 2H, J=8.0 Hz), 3.79 (s, 6H), 3.50 (t, 2H, J=8.2 Hz), 3.41-3.36 (m, 2H), 2.26 (t, 2H, J=7.6 Hz)

### [Preparation Example C] Preparation of Compound C

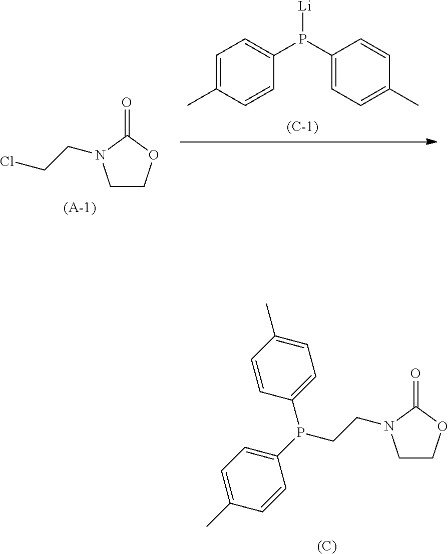

Compound C was obtained (yield: 41%) in the same manner as in Preparation Example A, except that Compound C-1 was used instead of Compound A-2.

¹H NMR (CDCl³, 500 MHZ) δ 7.34-7.31 (m, 4H), 7.16 (d, 4H, J=7.6 Hz), 4.18 (t, 2H, J=8.1 Hz), 3.51 (t, 2H, J=7.8 Hz), 3.43-3.38 (m, 2H), 2.34 (s, 6H), 2.30 (t, 2H, J=7.4 Hz)

### [Preparation Example 1] Preparation of Ligand Compound 1

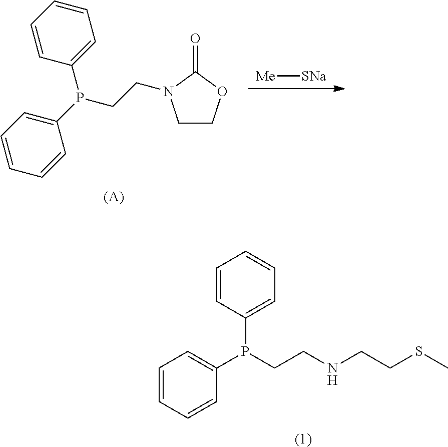

Compound A (Preparation Example A (2.99 g, 10 mmol)), NaS-Me (0.70 g, 10 mmol), Et₃N (1.39 mL, 10 mmol), and 20 mL of 2-methyl-2-butanol were added to a flask, and refluxed for 3 hours. The reactant was distilled under reduced pressure, and column purified to obtain Ligand Compound 1 (yield: 67%).

¹H NMR (CDCl₃, 500 MHZ) δ 7.45-7.41 (m, 4H), 7.34-7.32 (m, 6H), 2.80-2.74 (m, 4H), 2.61 (t, 2H, J=6.6 Hz), 2.28 (t, 2H, J=7.5 Hz), 2.08 (s, 3H), 1.57 (br, 1H)

### [Preparation Example 2] Ligand Compound 2

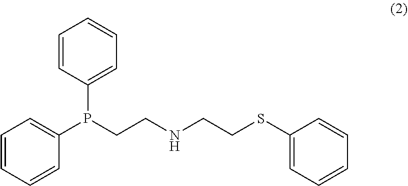 [SMILES: c1ccc(SCCNCCP(c2ccccc2)c2ccccc2)cc1]

Ligand Compound 2 was purchased from Strem Chemicals, Inc.

### [Preparation Example 3] Ligand Compound 3

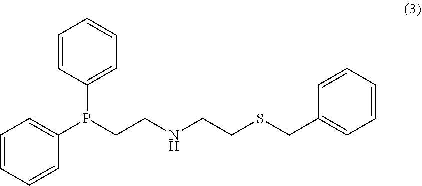 [SMILES: c1ccc(CSCCNCCP(c2ccccc2)c2ccccc2)cc1]

Ligand Compound 3 was purchased from Strem Chemicals, Inc.

### [Preparation Example 4] Ligand Compound 4

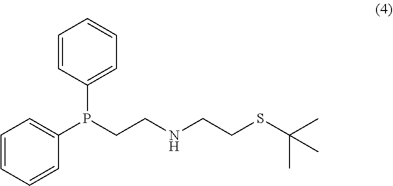 [SMILES: CC(C)(C)SCCNCCP(c1ccccc1)c1ccccc1]

Ligand Compound 4 was purchased from Strem Chemicals, Inc.

### [Preparation Example 5] Preparation of Ligand Compound 5

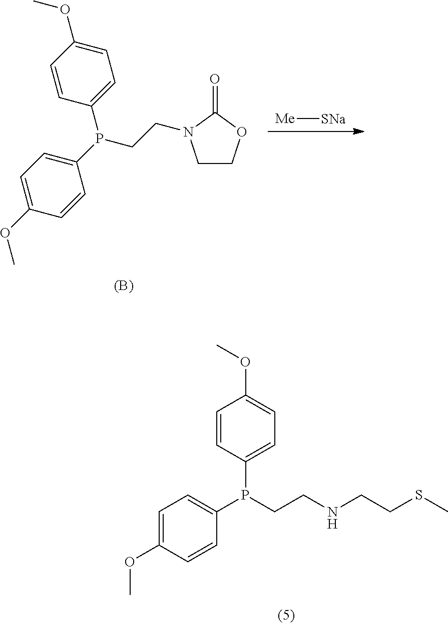

Ligand Compound 5 was obtained (yield: 17%) in the same manner as in Preparation Example 1, except that Compound B (Preparation Example B) was used instead of Compound A (Preparation Example A).

¹H NMR (CDCl₃, 500 MHZ) δ 7.37-7.34 (m, 4H), 6.87 (d, 4H, J=8.9 Hz), 3.80 (s, 6H), 2.79 (t, 2H, J=6.5 Hz), 2.77-2.72 (m, 2H), 2.61 (t, 2H, 6.6 Hz), 2.21 (t, 2H, 7.2 Hz), 2.01 (s, 3H), 1.72 (br, 1H)

### [Preparation Example 6] Preparation of Ligand Compound 6

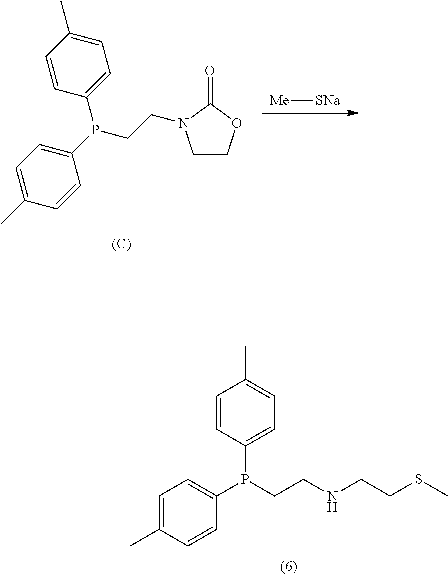

Ligand Compound 6 was obtained (yield: 32%) in the same manner as in Preparation Example 1, except that Compound C (Preparation Example C) was used instead of Compound A (Preparation Example A).

¹H NMR (CDCl₃, 500 MHZ) δ 7.32 (t, 4H, J=7.6 Hz), 7.14 (d, 4H, J=7.6 Hz), 2.81-2.74 (m, 4H), 2.62 (t, 2H, J=6.6 Hz), 2.33 (s, 6H), 2.26 (t, 2H, 7.4 Hz), 2.17 (br, 1H), 2.07 (s, 3H)

### [Preparation Example 7] Preparation of Ligand Compound 7

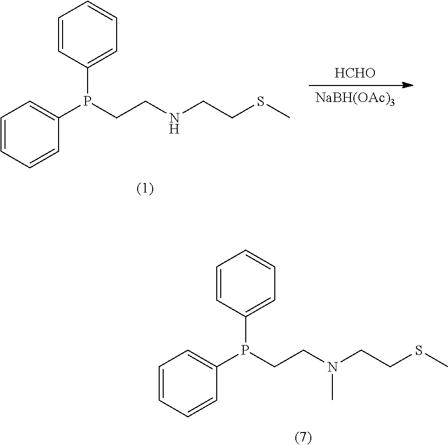 [SMILES: CSCCNCCP(c1ccccc1)c1ccccc1 ; "HCHO" ; "(1)" ; CSCCN(C)CCP(c1ccccc1)c1ccccc1 ; "(7)" ; "NaBH(OAc)3"]

Ligand Compound 1 (Preparation Example 1 (151 mg, 0.5 mmol)) and HCHO (30 mg, 1.0 mmol) were dissolved in 1.2 mL of dichloromethane in a flask, and stirring was performed for 15 minutes. NaBH(OAc)₃ (212 mg, 1.0 mmol) was slowly added, and refluxed for 24 hours. The reactant was distilled under reduced pressure, and column purified to obtain Ligand Compound 7 (yield: 48%).

¹H NMR (CDCl₃, 500 MHZ) 8 7.45-7.41 (m, 4H), 7.35-7.31 (m, 6H), 2.80-2.60-2.50 (m, 6H), 2.27 (s, 3H), 2.26-2.22 (m, 2H), 2.09 (s, 3H)

### [Comparative Preparation Example 1] Preparation of Ligand Compound 8

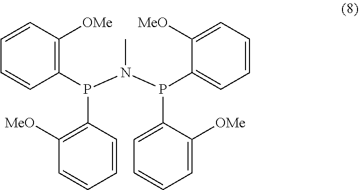 [SMILES: COc1ccccc1P(c1ccccc1OC)N(C)P(c1ccccc1OC)c1ccccc1OC]

Ligand Compound 8 was prepared with reference to a known literature (Organometallics 2001, 20, 4769-4771).

### [Example 1] Preparation of Oligomerization Catalyst 1

Chromium (III) acetylacetonate (Cr(acac)₃) (175 mg, 0.52 mmol) and 1.04 equiv. of Ligand Compound 1 (Preparation Example 1) were dissolved in 5 mL of toluene, and stirring was performed for 20 minutes. A liquid filtered using a 0.45 μm syringe filter was dried under vacuum to obtain a solid, which was dried under vacuum to obtain a solid (yield: 85%).

¹H NMR (C₆D₆, 500 MHZ) δ 3.58 (br), 1.41 (br)

### [Example 2] Preparation of Oligomerization Catalyst 2

Chromium (III) chloride tetrahydrofuran CrCl₃(THF)₃) (276 mg, 0.52 mmol) and 1.04 equiv. of Ligand Compound 1 (Preparation Example 1) were dissolved in 5 mL of tetrahydrofuran, and stirring was performed for 20 minutes. The formed precipitate was filtered using a filter, and a solid washed with diethyl ether was dried under vacuum to obtain a product (yield: 99%).

¹H NMR (DMSO, 500 MHZ) δ 8.84 (br), 5.06 (br), 3.97 (br), 2.07 (br)

### Example 3

Chromium (III) acetylacetonate (Cr(acac)₃) (6.99 mg, 20 μmol) and 1.2 equiv. of Ligand Compound 1 (Preparation Example 1) were dissolved in 4.12 mL of toluene, and stirring was performed for 10 minutes.

The dispersed mixed solution stirred in a glove box was added to a 20 mL autoclave reactor washed with nitrogen and vacuum. 0.86 mL of MMAO-12 (10% in toluene, Aldrich) (2 mmol, 100 equiv. of a chromium catalyst) and 20 μL of nonane were added, and then stirring was performed at 800 rpm. The temperature in the autoclave reactor was heated to 50° C., ethylene was filled to 20 bar, and an oligomerization reaction was performed for 30 minutes. After the reaction was completed, an excessive amount of ethylene in the reactor was discharged, the reactor was cooled to 10° C. or lower, the reactant was discharged to a discharge container containing 2.5 mL of methanol, and a small amount of an organic layer sample was analyzed by GC-FID. The remaining organic layer was filtered to separate a solid wax and a polymer product. The solid product was dried in an oven to measure the weight. The product of Example 3 after GC analysis is shown in Table 2.

### Examples 4 to 10

Products of Examples 4 and 10 were obtained in the same manner as in Example 3, except that the temperature, the pressure, the moles of the catalyst, and the MMAO equivalents for the catalyst were changed to the conditions shown in the following Table 1, and the results of analyzing the products by GC are shown in Table 2.

TABLE 1

| | | Ligand | | | |
|---|---|---|---|---|---|
| | | Compound 1 | | | |
| | | (Preparation | | | Temper- |
| | Cr(acac)₃ | Example 1) | MMAO | | ature |
| | (μmol) | (equiv.) | (equiv.) | Pressure(bar) | (° C.) |
| Example 3 | 20 | 1.2 | 100 | 20 (closed) | 50 |
| Example 4 | 20 | 1.2 | 100 | 20 (flow) | 70 |
| Example 5 | 20 | 1.2 | 100 | 20 (flow) | 100 |
| Example 6 | 10 | 1.2 | 100 | 20 (flow) | 100 |
| Example 7 | 2 | 1.2 | 100 | 20 (flow) | 100 |
| Example 8 | 0.1 | 1.2 | 100 | 20 (flow) | 100 |
| Example 9 | 2 | 1.2 | 100 | 30 (flow) | 100 |
| Example | 2 | 1.2 | 300 | 20 (flow) | 100 |
| 10 | | | | | |

TABLE 2

| | |
|---|---|
| Activity(g/g- | Selectivity(%) |
| Cr/h) for | in oligomer |

| | oligomers | 1-Hexene | 1-Octene | Etc. |
|---|---|---|---|---|
| Example 3 | 179.7 | 92.4 | 5.2 | 2.4 |
| Example 4 | 525.4 | 96.6 | 0.5 | 2.9 |
| Example 5 | 803.4 | 94.1 | 1.3 | 4.6 |
| Example 6 | 2135.2 | 93.7 | 1.3 | 5.0 |
| Example 7 | 10934.3 | 96.8 | 0.2 | 3.0 |
| Example 8 | 1322.8 | 80.1 | — | 19.9 |
| Example 9 | 5995.1 | 97.0 | 0.3 | 2.7 |
| Example 10 | 11820.2 | 95.5 | 1.3 | 3.2 |

As shown in Table 2, it was confirmed that oligomerization of ethylene employing Ligand Compound 1 (Preparation Example 1) showed excellent selectivity to 1-hexene, and the catalytic activity was very high even at a high temperature of 100° C. Activity of the oligomerization catalyst, selectivity to an oligomer, and selectivity to 1-hexene according to the change of the ligand compound were confirmed in the following example, by adopting the conditions of Example 7, which had excellent catalytic activity even with the use of a small amount of a chromium compound, among Examples 3 to 10.

### Example 11

Chromium (III) acetylacetonate (Cr(acac)₃) (0.7 mg, 2 μmol) and 1.2 equiv. of Ligand Compound 1 (Preparation Example 1) were dissolved in 4.12 mL of toluene, and stirring was performed for 10 minutes.

The dispersed mixed solution stirred in a glove box was added to a 20 mL autoclave reactor washed with nitrogen and vacuum. 0.86 mL of MMAO-12 (10% in toluene, Aldrich) (2 mmol, 100 equiv. of a chromium catalyst) and 20 μL of nonane were added, and then stirring was performed at 800 rpm. The temperature in the autoclave reactor was heated to 100° C., ethylene was filled to 20 bar, and an oligomerization reaction was performed for 30 minutes. After the reaction was completed, an excessive amount of ethylene in the reactor was discharged, the reactor was cooled to 10° C. or lower, the reactant was discharged to a discharge container containing 2.5 mL of methanol, and a small amount of an organic layer sample was analyzed by GC-FID. The remaining organic layer was filtered to separate a solid wax and a polymer product. The solid product was dried in an oven to measure the weight. The results of the product of Example 11 after GC analysis are shown in Table 3.

### Examples 12 to 17

The products of Examples 12 to 17 were obtained in the same manner as in Example 11, except that each of Ligand Compounds 2 to 6 (Preparation Examples 2 to 6) was used instead of using Ligand Compound 1 (Preparation Example 1), and the results of analyzing the products of Examples 12 to 17 by GC are shown in Table 3.

### Example 18

The compound of Example 1 (Oligomerization Catalyst 1) (1.2 mg, 2 μmol) was dissolved in 4.12 mL of toluene, and then stirring was performed for 10 minutes.

The dispersed mixed solution stirred in a glove box was added to a 20 mL autoclave reactor washed with nitrogen and vacuum. 0.86 mL of MMAO-12 (10% in toluene, Aldrich) (2 mmol, 100 equiv. of a chromium catalyst) and 20 μL of nonane were added, and then stirring was performed at 800 rpm. The temperature in the autoclave reactor was heated to 100° C., ethylene was filled to 20 bar, and an oligomerization reaction was performed for 30 minutes. After the reaction was completed, an excessive amount of ethylene in the reactor was discharged, the reactor was cooled to 10° C. or lower, the reactant was discharged to a discharge container containing 2.5 mL of methanol, and a small amount of an organic layer sample was analyzed by GC-FID. The remaining organic layer was filtered to separate a solid wax and a polymer product. The solid product was dried in an oven to measure the weight. The results of the product of Example 18 after GC analysis are shown in Table 3.

### Example 19

The compound of Example 2 (Oligomerization Catalyst 2) (0.9 mg, 2 μmol) was dissolved in 4.12 mL of toluene, and then stirring was performed for 10 minutes.

The dispersed mixed solution stirred in a glove box was added to a 20 mL autoclave reactor washed with nitrogen and vacuum. 0.86 mL of MMAO-12 (10% in toluene, Aldrich) (2 mmol, 100 equiv. of a chromium catalyst) and 20 μL of nonane were added, and then stirring was performed at 800 rpm. The temperature in the autoclave reactor was heated to 100° C., ethylene was filled to 20 bar, and an oligomerization reaction was performed for 30 minutes. After the reaction was completed, an excessive amount of ethylene in the reactor was discharged, the reactor was cooled to 10° C. or lower, the reactant was discharged to a discharge container containing 2.5 mL of methanol, and a small amount of an organic layer sample was analyzed by GC-FID. The remaining organic layer was filtered to separate a solid wax and a polymer product. The solid product was dried in an oven to measure the weight. The results of the product of Example 19 after GC analysis are shown in Table 3.

### Comparative Example 1

The product of Comparative Example 1 was obtained in the same manner as in Example 11, except that Ligand Compound 8 (Comparative Preparation Example 1) was used instead of using Ligand Compound 1 (Preparation Example 1), and the results of analyzing the product by GC are shown in Table 3.

TABLE 3

| | | | Activity | | | Selectivity in | | |
|---|---|---|---|---|---|---|---|---|
| | | | (g/g- | | | oligomer (%) | | |
| | Chromium | Ligand | Cr/h) for | Selectivity (%) | | 1- | 1- | |
| | compound | compound | oligomers | Polymer | Oligomer | Hexene | Octene | Etc. |
| Example 11 | Cr (acac)₃ | 1 | 10934.3 | 1.9 | 98.1 | 96.8 | 0.2 | 3.0 |
| Example 12 | Cr (acac)₃ | 2 | 786.1 | 11.3 | 88.7 | 91.0 | 4.0 | 5.0 |
| Example 13 | Cr (acac)₃ | 3 | 95.4 | 19.5 | 80.5 | 74.9 | 3.2 | 21.9 |
| Example 14 | Cr (acac)₃ | 4 | 55.7 | 29.0 | 71.0 | 74.5 | — | 25.5 |
| Example 15 | Cr (acac)₃ | 5 | 6060.0 | 5.4 | 94.6 | 96.2 | 0.5 | 3.3 |
| Example 16 | Cr (acac)₃ | 6 | 11130.5 | 1.2 | 98.8 | 96.2 | 0.3 | 3.5 |
| Example 17 | Cr (acac)₃ | 7 | 3471.9 | 7.7 | 92.3 | 95.1 | 0.7 | 4.2 |
| Example 18 | Example 1 | | 1376.0 | 18.6 | 81.4 | 63.0 | 7.9 | 29.1 |
| | (Oligomerization | | | | | | | |
| | Catalyst 1) | | | | | | | |
| Example 19 | Example 2 | | 1924.0 | 2.9 | 97.1 | 98.6 | 0.2 | 1.2 |
| | (Oligomerization | | | | | | | |
| | Catalyst 2) | | | | | | | |
| Comparative | Cr (acac)₃ | 8 | 2910.5 | 85.2 | 14.8 | 71.2 | 2.9 | 25.9 |
| Example 1 | | | | | | | | |

As shown in Table 3, in Examples 11 to 17 using Ligand Compounds 1 to 7 of the present invention, selectivity to an oligomer was 70 or more, and thus, significantly excellent selectivity was in the examples of the present invention, as compared with the oligomer selectivity of Comparative Example 1 using conventionally used Ligand Compound 8, which was 14.8%, and in particular, the selectivities of the oligomer of Examples 11 and 16 were 98% or more, which showed excellent selectivity.

In addition, the example of the present invention had excellent selectivity to 1-hexene, and in particular, Examples 11, 12, 15, 16, 17, and 19 had surprisingly improved selectivity to 1-hexene of 90% or more, and thus, it was confirmed that the oligomerization catalyst and the catalyst composition including the ligand compound of the present invention were very efficient in the preparation of 1-hexene.

The oligomerization catalyst of the present invention maintains catalytic activity and selectivity even at a high temperature, produces less by-products to cause no problem of pipe blockage and fouling, does not need shutdown of a polymerization process, and thus, is very economical. Furthermore, since the oligomerization catalyst of the present invention has excellent catalytic activity even at a high temperature, an oligomer may be prepared with only a small amount of the catalyst and a small amount of the organoaluminum compound, the activity is not lowered even at a high temperature, selectivity is excellent, and thus, 1-hexene may be prepared from ethylene with surprisingly excellent selectivity.

1. An ethylene oligomerization catalyst comprising a chromium compound and a ligand compound represented by the following Chemical Formula 1: 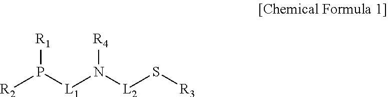
wherein
L₁ and L₂ are independently of each other (C2-C4)alkylene;
R₁ to R₄ are independently of one another hydrogen, (C1-C10)alkyl, (C3-C20)cycloalkyl, (C6-C20)aryl, (C6-C20)aryl(C1-C10)alkyl, or (C1-C10)alkoxy; and
the aryl of R₁ to R₄ may be substituted by one or two or more substituents selected from the group consisting of (C1-C10)alkyl, (C6-C20)aryl, tri(C1-C10)alkylsilyl, and (C1-C10)alkoxy.

2. The ethylene oligomerization catalyst of claim 1, wherein in Chemical Formula 1,
L₁ and L₂ are independently of each other (C2-C3)alkylene;
R₁ to R₄ are independently of one another hydrogen, (C1-C7)alkyl, (C3-C12)cycloalkyl, (C6-C12)aryl, (C6-C12)aryl(C1-C7)alkyl, or (C1-C7)alkoxy; and
the aryl of R₁ to R₄ may be substituted by one or two or more substituents selected from the group consisting of (C1-C7)alkyl, (C6-C12)aryl, tri(C1-C7)alkylsilyl, and (C1-C7)alkoxy.

3. The ethylene oligomerization catalyst of claim 1, wherein in Chemical Formula 1,
L₁ and L₂ are independently of each other (C2-C3)alkylene;
R₁ to R₄ are independently of one another hydrogen, (C1-C7)alkyl, (C6-C12)aryl, or (C6-C12)aryl(C1-C7)alkyl; and
the aryl of R₁ to R₄ may be substituted by one or two or more substituents selected from the group consisting of (C1-C7)alkyl, (C6-C12)aryl, tri(C1-C7)alkylsilyl, and (C1-C7)alkoxy.

4. The ethylene oligomerization catalyst of claim 1, wherein the ligand compound is represented by the following Chemical Formula 2: 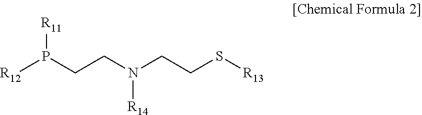
wherein
R₁₁ to R₁₄ are independently of one another hydrogen, (C1-C5)alkyl, (C6-C10)aryl, or (C6-C10)aryl(C1-C5)alkyl; and
the aryl of R₁₁ to R₁₄ may be substituted by one or two or more substituents selected from the group consisting of (C1-C5)alkyl, tri(C1-C5)alkylsilyl, and (C1-C5)alkoxy.

5. The ethylene oligomerization catalyst of claim 4, wherein in Chemical Formula 2,
R₁₁ to R₁₃ are independently of one another (C1-C5)alkyl, (C6-C10)aryl, or (C6-C10)aryl(C1-C5)alkyl;
R₁₄ is hydrogen or (C1-C3)alkyl; and
the aryl of R₁₁ to R₁₃ may be substituted by one or two or more substituents selected from the group consisting of (C1-C5)alkyl, tri(C1-C5)alkylsilyl, and (C1-C5)alkoxy.

6. The ethylene oligomerization catalyst of claim 1, wherein the ligand compound is selected from the following compounds:

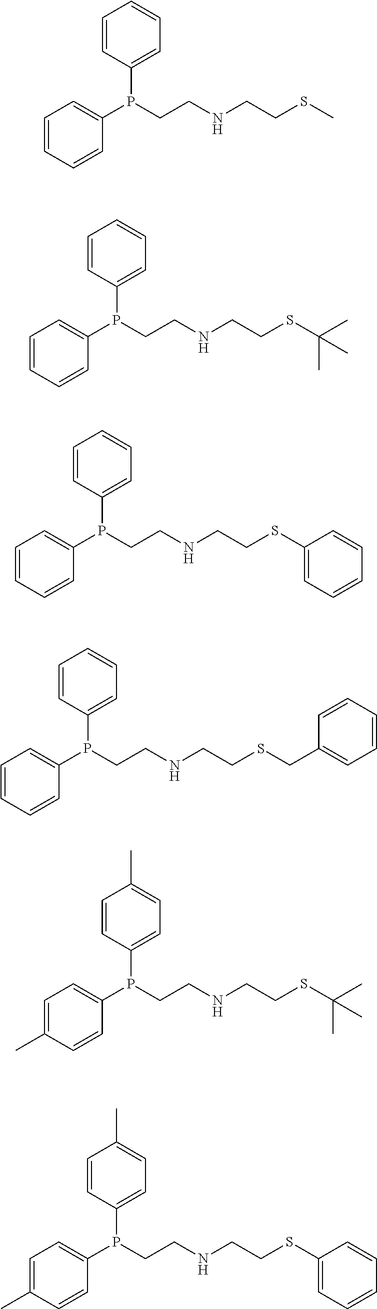

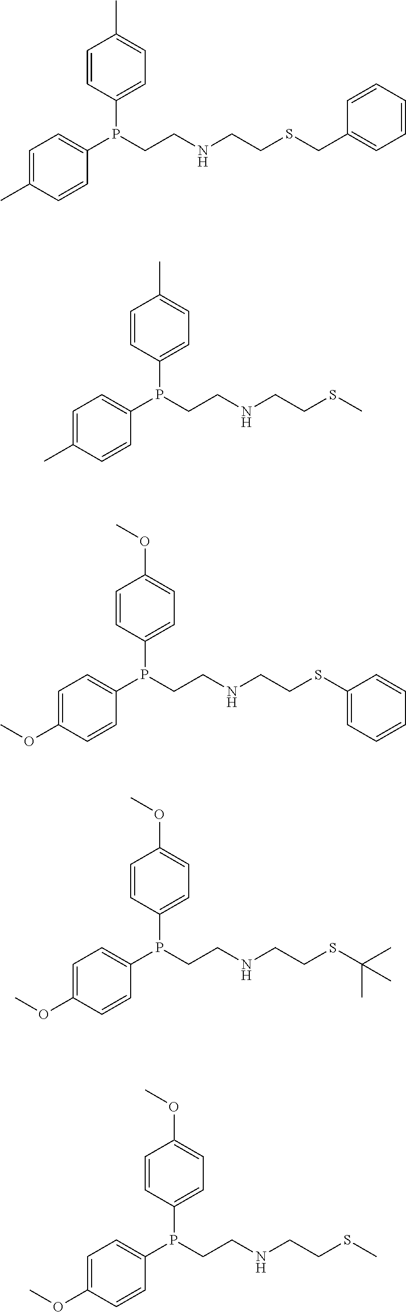

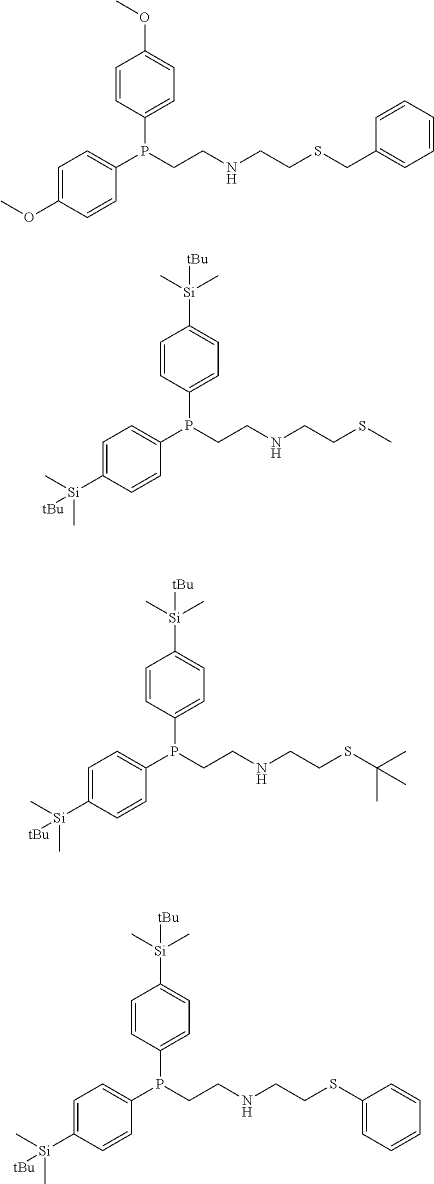

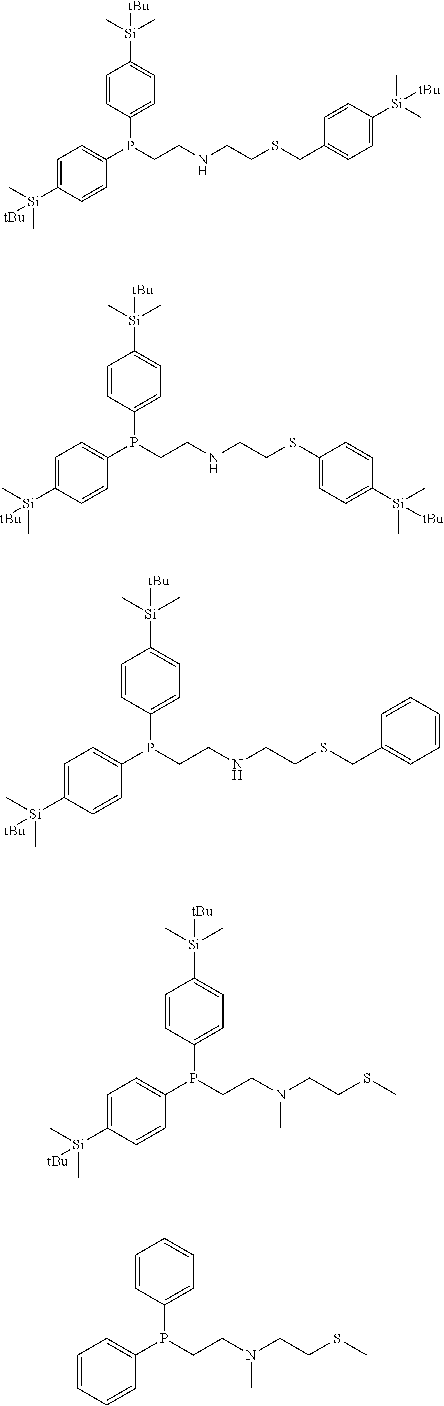

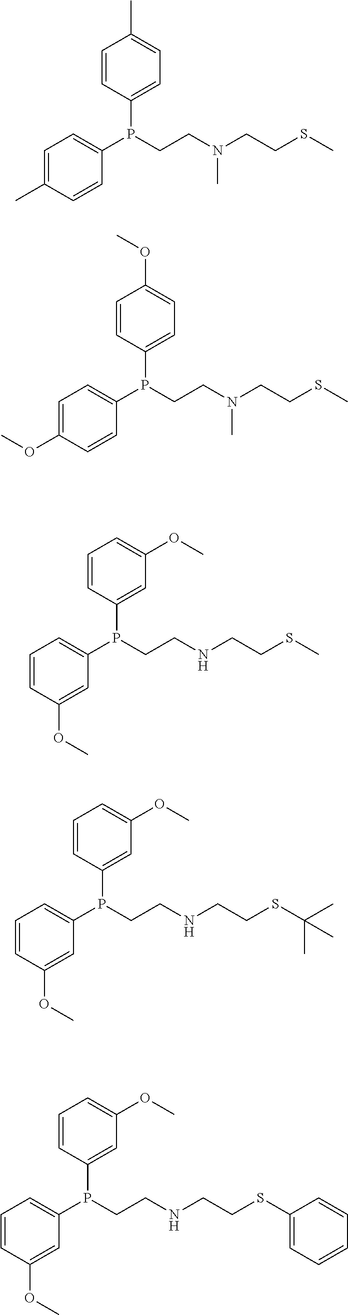

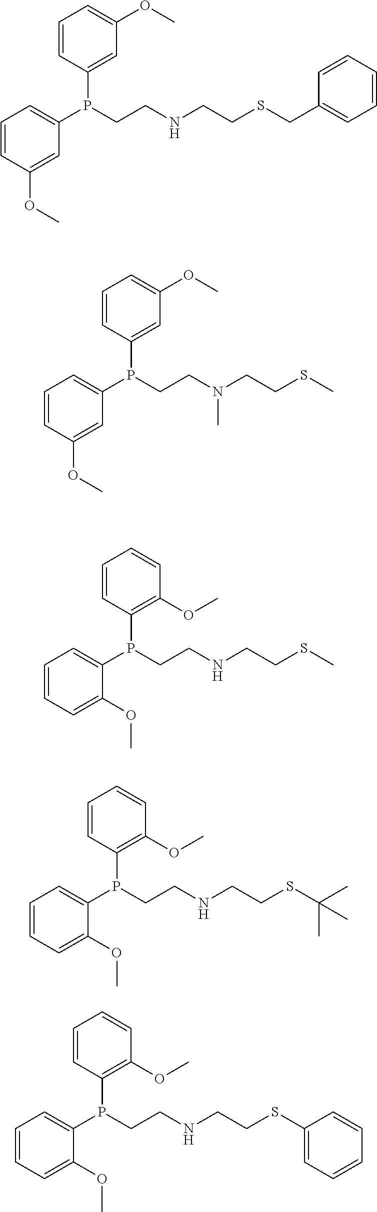

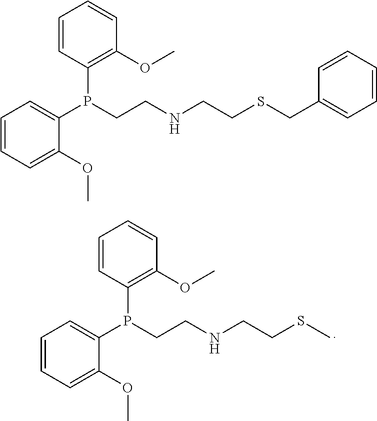

7. An ethylene oligomerization catalyst composition comprising the ethylene oligomerization catalyst of claim 1 an organoaluminum compound.

8. The ethylene oligomerization catalyst composition of claim 7, wherein the organoaluminum compound is one or two or more selected from the group consisting of methylaluminoxane (MAO), modified methylaluminoxane (MMAO), ethylaluminoxane (EAO), tetraisobutylaluminoxane (TIBAO), isobutylaluminoxane (IBAO), trimethylaluminum (TMA), triethylaluminum (TEA), triisobutylaluminum (TIBA), tri-n-octylaluminum, methylaluminum dichloride, ethylaluminum dichloride, dimethylaluminum chloride, diethylaluminum chloride, aluminum isopropoxide, ethylaluminum sesquichloride, and methylaluminum sesquichloride.

9. A method for preparing an ethylene oligomer using the ethylene oligomerization catalyst composition of claim 7.

10. The method for preparing an ethylene oligomer of claim 9, wherein 60.0 to 99.999 wt % of 1-hexene of the total oligomer is prepared.

11. The method for preparing an ethylene oligomer of claim 9, wherein the method for preparing an ethylene oligomer is performed in one or two or more solvents selected from the group consisting of benzene, chlorobenzene, ethylbenzene, toluene, xylene, cumene, mesitylene, cyclohexane, methylcyclohexane, methylcyclopentane, hexane, heptane, octane, nonane, decane, hexene, heptene, octene, nonene, decene, anisole, ethoxybenzene, and dimethoxybenzene.
